(19) United States
(12) Patent Application Publication
(10) Pub. No.: US 2023/0321417 A1
(43) Pub. Date: Oct. 12, 2023

(54) Automated External Defibrillator with Integrated Medication Delivery

(71) Applicant: HeartHero, Inc., Denver, CO (US)

(72) Inventors: Gary Montague, Denver, CO (US); Anthony Verdeja, Denver, CO (US)

(21) Appl. No.: 18/297,766

(22) Filed: Apr. 10, 2023

Related U.S. Application Data
(60) Provisional application No. 63/329,103, filed on Apr. 8, 2022.

(51) Int. Cl. A61M 35/00; A61N 1/39; A61N 1/04

(52) U.S. Cl. CPC A61M 35/006; A61N 1/3904; A61N 1/046; A61N 1/0496; A61M 2205/054; A61M 2037/0023

(57) ABSTRACT

An automated external defibrillator (AED) system includes shock generating electronics configured to provide at least one electrical shock suitable for a patient experiencing a cardiac event, a battery configured for providing power to the shock generating electronics, power management circuitry configured for managing the shock generating electronics and the battery, a single microprocessor configured for controlling the power management circuitry, and an enclosure configured to house the shock generating electronics, the battery, the power management circuitry, and the single microprocessor. In an embodiment, the AED system includes at least two cardiac pads in electrical connection with the shock generating electronics and including a medication delivery mechanism configured for delivering a predetermined dose of a medication to a patient when the cardiac pads are placed on the patient for shock delivery.

### CROSS REFERENCE TO RELATED APPLICATIONS

The present application claims priority to U.S. Provisional Patent Application No. 63/329,103, filed Apr. 8, 2022 and titled “Automated External Defibrillator.”

The present application is also related to U.S. patent application Ser. No. 15/847,826 filed Dec. 19, 2017, now U.S. Pat. No. 11,103,718, entitled “Automated External Defibrillator Device and Methods of Use,” and U.S. patent application Ser. No. 17/548,193, filed Dec. 10, 2021, now U.S. Pat. No. 11,529,526, and titled “Automated External Defibrillator.”

All of the aforementioned patent applications are incorporated herein by reference in their entirety.

### FIELD OF THE DISCLOSURE

Aspects of the present disclosure generally relate to automated external defibrillators (AEDs) and, more particularly, to compact AED systems and methods.

### BACKGROUND OF THE DISCLOSURE

86 million Americans have risk factors for sudden cardiac arrest (SCA), while 12 million are at high risk. Cardiac events represent more deaths in America than breast, lung, colon and prostate cancer combined. More than 360,000 SCAs occur outside of the hospital each year. According to the American Heart Association, nearly 70 percent of these SCAs occur at home, out of reach of the lifesaving shock of an AED.

As each minute passes following a SCA, the chances of survival decrease significantly. If an AED is not applied within 10 minutes of a SCA event, chances of survival decrease to less than 1%.

One approach to increasing the chance of survival for SCA sufferers is to make AEDs more readily available and accessible for more people. However, the AEDs currently available on the market tend to be heavy, not portable, expensive, and intimidating to use for people without medical training. For example, U.S. Pat. No. 11,103,718, entitled “Automatic External Defibrillator Device and Methods of Use,” which disclosure is incorporated herein in its entirety by reference, provides a possible solution to overcome the availability and accessibility problem by providing a compact AED device suitable for portability.

Additionally, due to the prevalence of opioid addiction around the world and in the United States, the US Department of Health and Human Services has declared a public health emergency in 2017. It is recognized herein that often times cardiac distress is accompanied by other emergency conditions, such as opioid overdose, that also must be addressed as quickly as possible by emergency responders.

Aspects of the present disclosure provide techniques and structures that improve the performance of AEDs suitable for high portability applications.

### SUMMARY OF THE DISCLOSURE

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

In some aspects, the techniques described herein relate to an automated external defibrillator (AED) system, including: shock generating electronics configured to provide at least one electrical shock suitable for a patient experiencing a cardiac event; a battery configured for providing power to the shock generating electronics; power management circuitry configured for managing the shock generating electronics and the battery; a single microprocessor configured for controlling the power management circuitry; and an enclosure configured to house the shock generating electronics, the battery, the power management circuitry, and the single microprocessor. In an embodiment, the AED system includes two cardiac pads in electrical connection with the shock generating electronics. At least one of the two cardiac pads includes a medication delivery mechanism configured for delivering a predetermined dose of a medication to a patient when the cardiac pads are placed on the patient for shock delivery. The medication delivery mechanism includes, for example, a medication-impregnated transdermal patch, a microneedle array, or the like. In certain embodiments, the medication includes naloxone.

Optionally, in certain aspects, the AED system includes a clip mounted to an exterior of the enclosure, wherein the clip is configured for clipping the AED system to a user's belt or another location such as a bag for carrying the AED system.

In embodiments, the AED system further includes a plurality of pairs of cardiac pads. Each one of the plurality of pairs of cardiac pads may be configured for electrical connection with the shock generating electronics one at a time, and each one of the plurality of pairs of cardiac pads includes a different predetermined dose of medication from each other one of the plurality of pairs of cardiac pads.

In certain embodiments, at least one of the cardiac pads in the pair of cardiac pads includes a medication identification feature for identifying the medication integrated thereon. In some embodiments, the medication identification feature includes at least one of a radio frequency identification (RFID) chip, a near infrared (NIR) chip, an identification circuit, a liner label, an electrode protector label, an adhesive label, and a package label.

In some aspects, the techniques described herein relate to a method for using an external defibrillator (AED) system to assist a patient in cardiac distress, the AED system including shock generating electronics, a single battery configured for providing power to the shock generating electronics, power management circuitry configured for managing the shock generating electronics and the battery, a single microcontroller configured for controlling the power management circuitry, an enclosure, and a pair of cardiac pads including a medication delivery mechanism. The method includes: applying the pair of cardiac pads onto the patient; automatically delivering a medication to the patient via the medication delivery mechanism on the cardiac pads; monitoring a charge status of the battery of the AED; monitoring vital signs via the cardiac pads; charging the shock generating electronics; determining whether a shockable rhythm exists; and administering a shock to the patient via the cardiac pads when the shockable rhythm exists.

### BRIEF DESCRIPTION OF THE DRAWINGS

Embodiments of the invention are described in detail below with reference to the attached drawing figures, wherein:

FIG. 1 illustrates a schematic of an AED system, in accordance with an embodiment.

FIG. 2 illustrates a block diagram of an exemplary AED, including an AED operations block and a communications block, in accordance with an embodiment.

FIG. 3A illustrates a front view of an automated external defibrillator (AED), in accordance with an embodiment.

FIG. 3B illustrates a perspective view of the AED, in accordance with an embodiment.

FIG. 4 illustrates a flow diagram of a method configured for use with any of the AEDs of FIGS. 1-3B, in accordance with an embodiment.

FIGS. 5A-5E illustrate an exemplary embodiment of pads suitable for use with the AED system with medicine delivery.

FIG. 6 illustrates a flow diagram of a method configured for use with an AED with separately provided cardiac pads, including any of the AEDs of FIGS. 1-3B, in accordance with certain embodiments.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized, and changes can be made without departing from the scope of the invention. The following detailed description is, therefore, not to be taken in a limiting sense. The scope of the invention is defined only by the appended claims, along with the full scope of the equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. Described in one embodiment may also be included in other embodiments, although is not necessarily included. Thus, the technology can include a variety of combinations and/or integrations of the embodiments described herein.

If more AEDs can be made available to more people, with improved portability, lower cost, and enhanced ease of use, then more lives can be saved in the event of a cardiac event, such as a sudden cardiac arrest (SCA), occurring outside of a hospital setting. That is, like an EPIPEN® injector is prescribed for and carried by those diagnosed with potentially life-threatening allergies, a portable AED can be a necessary and routine item prescribed to those diagnosed as being at risk for SCA. A portable, affordable, and user-friendly AED with safe and simple application protocol is desired for such wide-spread proliferation of AEDs in the consumer market. Additionally, a device that is small and light enough to be worn or carried by emergency personnel is desired so that a life-saving shock may be administered in the field (e.g., prior to moving a patient to an ambulance).

Further, there are many instances where a patient in cardiac distress also requires simultaneous treatment for another condition. For example, cardiac events may accompany other health events, such as opioid overdose necessitating the administration of naloxone.

Particularly related to the treatment of a cardiac patient experiencing opioid overdose, various forms of naloxone delivery, such as injections and nasal sprays are available. However, the administration of the naloxone by these means to a patient in cardiac distress requires additional steps to be undertaken by the emergency responder, in addition to the essential procedures related to the administration of cardiopulmonary resuscitation (CPR) and, often, electrical shock from an AED.

Thus, it is recognized herein that combining the delivery of medicine within the required steps in the administration of lifesaving electrical shock from an AED can save time and likely lead to better outcomes. In particular, the embodiments described herein includes the incorporation of a medicine delivery mechanism into the cardiac pads used to deliver the electrical shock to the patient in cardiac distress. In this way, without additional steps, the emergency responder can administer the necessary medication within the ordinary procedural flow and at the same time as the AED shock delivery.

Optionally, a minimalist AED, such as that described in copending, co-owned U.S. patent application Ser. No. 17/548,193, mentioned above, may be provided with a variety of cardiac pads including different medications such that the emergency responder may select the necessary drug for specific cases. For instance, a minimalist AED with naloxone pads may be supplied to be carried by emergency personnel in areas known to have a high rate of opioid addiction, while AEDs provided at locations with reduced likelihood of opioid-related cardiac distress situations (e.g., senior care facilities and elementary schools) may be supplied with normal cardiac pads or pads imbued with a medication other than naloxone.

An exemplary embodiment of the AED includes: (1) a defibrillator including a battery to charge a capacitor to store and deliver an electric shock; (2) a communication module to transmit and receive data via a wireless connection; and (3) cardiac pads with electrodes to detect and monitor chest wall compression depth, compression rate, and chest wall impedance, and heart rhythm; and (4) a smartphone or mobile device application to analyze information received from the cardiac pads and recommend appropriate therapy, the application also having the ability to contact EMS via the smartphone/mobile device with GPS, Wi-Fi and/or cellular capabilities. In certain embodiments, these components are connected as follows: a smartphone with application is connected to the defibrillator via either a wired or wireless connection, such as Bluetooth® or Wi-Fi, then at least two electrodes with wires ending in cardiac pads connect from the battery/capacitor pack to the patient's chest.

Certain embodiments described herein include one or more of the following: (1) use one or more common household batteries that can be purchased off-the-shelf, in contrast with AEDs requiring specialized battery packs; (2) include specialized capacitors and circuitry that generate a therapeutic charge from the off-the-shelf battery; (3) continuously analyze the cardiac rhythm during CPR; (4) include sensors in the cardiac pads to detect impedance of the chest wall and ensure proper pad connection; (5) include additional sensors in the cardiac pad to monitor compression force, rate and depth of CPR; (6) by using the sensors to monitor vital signs, ensure that a cardiac shock is not given at an undesired time; and (7) via the sensors inside the cardiac pad, communicate information to the software system regarding size of chest wall which then allows for determination of a therapeutic shock that is correlated with the size of victim and their individual anatomy.

Furthermore, the processes associated with certain embodiments of the invention provide the following: (1) the AED is wearable so that an EMT or other trainer user may have the AED on their person at all times while on duty; (2) the cardiac pad and/or the packaging containing the cardiac pad with the medicine delivery mechanism may include an identifiable signature, such as a specific electrical profile, radio frequency identification (RFID) chip, near-infrared (NIR) chip, or other means such that the AED is able to identify, by hardware or software, the specific medication that has been imbued in a given pair of cardiac pads; (3) a software application analyzes cardiac rhythm and provides electric shock for appropriate cardiac arrhythmias; and (4) the user will be prompted to stop CPR upon return of spontaneous circulation (ROSC).

In an embodiment, a mobile device is connected via hardwire, Bluetooth® or Wi-Fi to a case that holds the battery, specialized capacitors, and circuitry. At least two cardiac pads with sensors are stored separately and connected via wire to the AED when needed. The case protects the user from the risk of electrical shock and protects the internal electronics from electrostatic discharge (ESD), which can cause the electronics to fail or malfunction in an unsafe way. Suitable materials for the case may include a variety of plastics and other insulating materials.

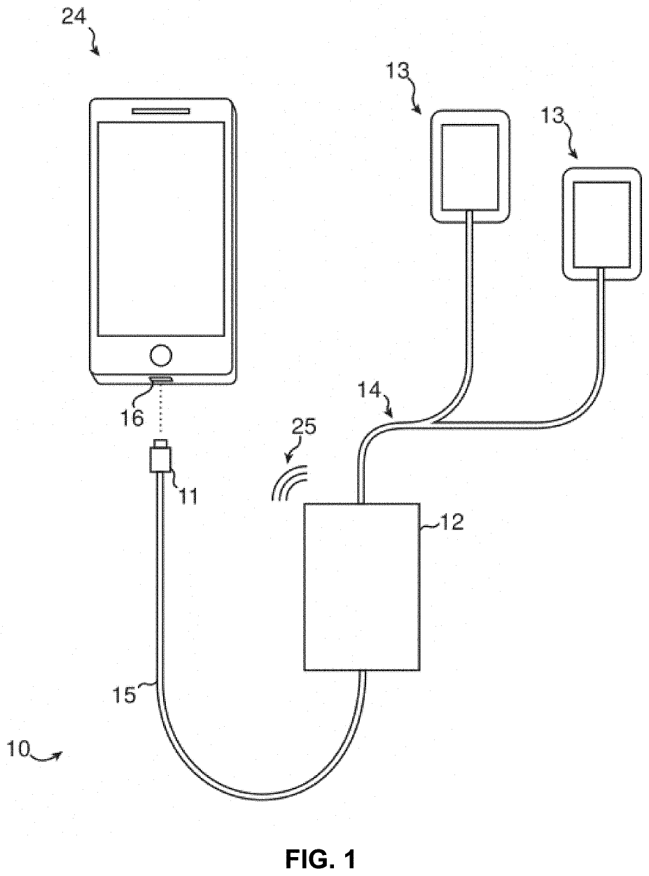

FIG. 1 shows a schematic of an AED system 10, in accordance with an embodiment. AED system includes a connector 11, an electronics module 12, at least two electro-conductive cardiac pads 13, and electrical conductors such as wiring 14 connecting cardiac pads 13 with electronics module 12. Cardiac pads 13 include sensors (not shown) for monitoring, for example, cardiac rhythm and body impedance of the SCA patient to whom cardiac pads 13 are connected. Further, as mentioned above, cardiac pads 13 include a medicine delivery mechanism, such as a peel-and-stick transdermal patch impregnated with a medicine, a microneedle array imbued with a known dose of a medication, or a blister pack containing a medication. Optionally or additionally, cardiac pads 13 may include an indicator, such as an identification chip, labels, color or alphanumeric codes, and other mechanism for providing a visual indication of the integration of the medicine delivery mechanism as well as the type of medication incorporated therein. Further, cardiac pads 13 and/or wiring 14 may provide an indication (e.g., an electronic signal, a specified impedance or capacitance) to enable electronics module 12 to automatically detect the integration of the medicine delivery mechanism in the cardiac pads connected thereto.

The sensors in cardiac pads 13 may also indicate whether cardiac pads 13 are properly placed on the SCA patient, and may indicate to electronics module 12 if one or both of cardiac pads 13 are disconnected from the SCA patient. Furthermore, sensors in cardiac pads 13 may also include capabilities such as detection of force, compression rate, and depth of compression to help monitor and provide feedback on any cardiopulmonary resuscitation (CPR) performed on the SCA patient. Connector 11 is attached to electronics module 12 via a wire 15 in the embodiment shown in FIG. 1. Alternatively, the connection between the mobile device and electronics module 12 is established wirelessly through, for instance, Bluetooth® or Wi-Fi. Connector 11 may optionally be attached via a receptacle 16 to a mobile device 24.

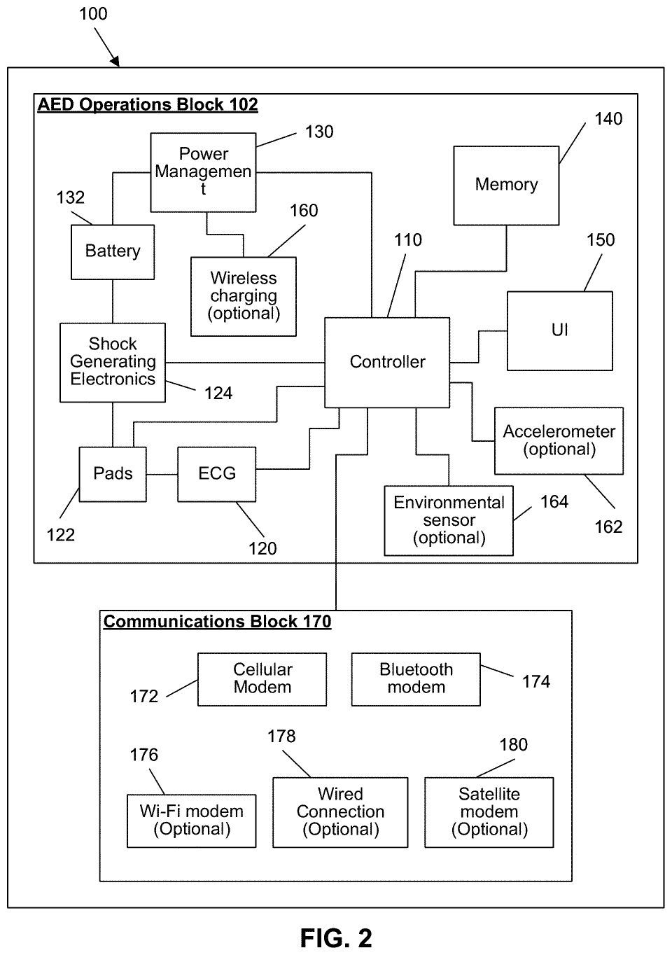

FIG. 2 is a block diagram of an exemplary AED 100 including an AED operations block 102 and a communications block 170, in accordance with an embodiment. AED operations block 102 includes various components that enable AED 100 to generate and deliver, within regulatory guidelines, an electric shock to a person experiencing a cardiac event such as SCA. As shown in the embodiment illustrated in FIG. 2, AED operations block 102 includes a controller 110, which controls a variety of components including an electrocardiogram (ECG) monitoring circuitry 120, which is in turn connected with cardiac pads 122. Cardiac pads 122 are configured for attachment to specific locations on a patient experiencing a cardiac event for delivering an electric shock to the patient. Further, cardiac pads 122 integrates therein a medicine delivery mechanism, such as a transdermal patch impregnated with a medicine, a microneedle array imbued with a known dose of a medication, or a blister pack containing a medication. In an embodiment, the medicine delivery mechanism is formed without metallic components that could interfere with effective delivery of AED shock to the patient, while enabling transdermal or subcutaneous delivery of the impregnated medicine to the patient at the same time as the AED shock procedure.

In embodiments, cardiac pads 122 are configured to receive ECG signals from the patient in addition to administering the electric shock. In some embodiments, cardiac pads 122 are waterproof. The cardiac pads 122 may be configured for reuse a predetermined number of times, after which the cardiac pads are replaced. In such cases, the medicine delivery mechanism in the cardiac pads may further include dosing mechanisms, such as multiple peel-off layers configured for delivering a single dose of the medication, where the old peel-off layer may be removed prior to attachment to a new patient to enable delivery of a fresh dose of medication to the new patient. The ECG signals are transmitted from cardiac pads 122 to ECG monitoring circuitry 120, which is communicatively coupled with controller 110.

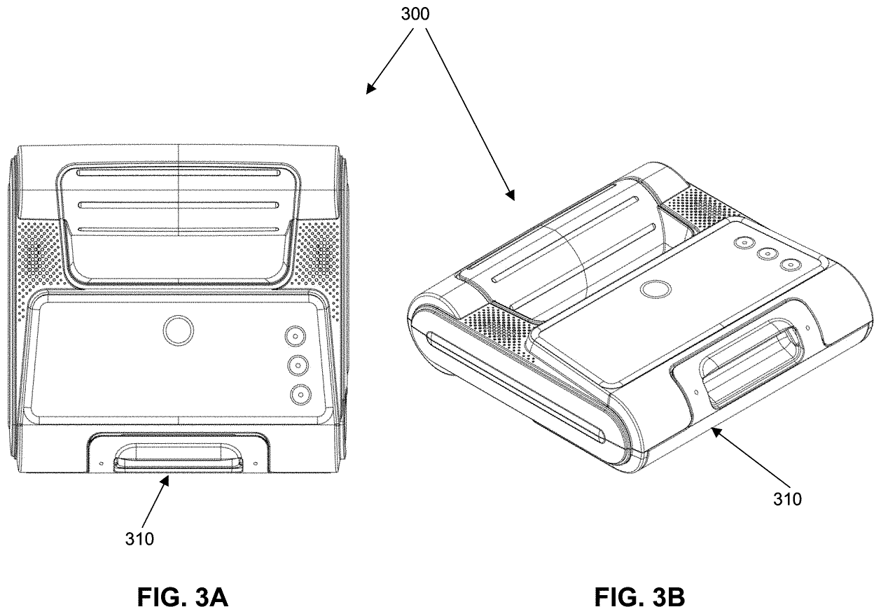

Shock generating electronics 124 are configured to charge a capacitor under control of controller 110. In embodiments, AED 100 includes a flat square capacitor for fitting within the case or enclosure of AED 100, while in other embodiments the charge storing capacitor may be customized to fit certain portions of the AED (e.g., a handle portion of AED 300 shown in FIG. 3). In embodiments, the shock generating electronics 124 are configured to provide a voltage waveform that is between approximately 120 and 200 Joules in total energy. In certain embodiments, the shock generating electronics 124 include a quad-phasic truncated exponential power stage that is configured to produce a shock suitable for defibrillation while reducing the complexity and size of the capacitor.

Controller 110 may include, for example, non-transitory memory for storing software instructions. The non-transitory memory may be communicatively coupled with a processor (e.g., microprocessor) for executing software instructions stored on the non-transitory memory. Software instructions may include, for instance, workflow information for operating AED 100, as described herein. Controller 110 is also connected with a memory 140, which stores information regarding AED 100, such as use history, battery status, shock administration and CPR protocols, and other information (e.g., stored in look-up tables) used in the operation of AED 100. Memory 140, may, in some embodiments, be used by controller 110 to instruct a user on CPR and/or shock administration via communications block 170. In embodiments, controller 110 is a single microcontroller or single microprocessor.

AED operations block 102 includes a power management block 130, which is also controlled by controller 110 in embodiments. Power management block 130 comprises power management circuitry configured for managing the power consumption by various components within AED operations block 102. For instance, power management block 130 monitors a charge status of a battery 132, which provides electrical power to shock generating electronics 124. Power management block 130 provides instructions for controlling the on/off status of all electrical components of AED 100 via controller 110 so as to minimize power consumption while AED 100 is not being used. For example, power management block 130 is configured to completely power down ECG monitoring circuitry 120 and shock generating electronics 124 when the AED is not being used. In embodiments, power management block 130 is configured to provide a signal to a user via controller 110 for notifying the user of a charge status of battery 132.

A user-interface (UI) 150 is communicatively coupled with controller 110. In embodiments, UI 150 provides a simple user interface that minimizes space and electrical power requirements. For example, UI block 150 intentionally omits a graphic user interface such as a liquid crystal display (LCD) or touchscreen in order to reduce the size and complexity of AED 100. UI 150 may include a speaker to provide audible sounds (e.g., beeps) and/or voice prompts for aiding the AED user.

In certain embodiments, the UI features may be eliminated to further reduce the size and weight of the AED.

Still referring to FIG. 2, AED 100 includes a communications block 170, also controlled by controller 110. Communications block 170 provides connections to external systems and entities outside of the AED, such as emergency medical services, hospital emergency rooms, physicians, electronic health record systems, as well as other medical equipment, such as ventilators and an external ECG. Communications block 170 includes at least one of the following communication features: a cellular modem 172, a Bluetooth® modem 174, a Wi-Fi modem 176 for providing wireless connection to and from an external device. The various communication modes within communications block 170 are configured to comply with regulatory guidance related to wireless technology, such as coexistence, security, and electromagnetic compatibility. By having a single controller (e.g., a microprocessor) control communications block 170 within AED 100, the circuit design and firmware configuration of AED 100 is greatly consolidated over other AEDs with multiple processors, while enabling a reduction in power consumption of the device.

FIGS. 3A and 3B illustrate front and perspective views, respectively, of a portable AED 300. Portable AED 300 includes a pads cartridge 310, which can be pulled out using an integrated handle. Alternatively, a minimalist AED, such as that described in the aforementioned U.S. patent application Ser. No. 17/548,193 with an externally supplied cardiac pads may be advantageous for situations requiring high portability in use by medical professionals. For instance, in some embodiments, the AED is configured for wearing or carrying by a trained user without having cardiac pads connected. Without the pads connected, the AED has a smaller profile and avoids having exposed wiring connected to the AED while the device is worn or carried, which prevents the wires from getting caught or tangled. Additionally, in certain embodiments, the AED lacks a storage compartment for cardiac pads so as to reduce the overall size of the device. The cardiac pads are therefore carried separately by the user.

Alternatively, portable AED 300 may be provided with a connector port into which a separately provided cardiac pads arrangement may be connected by emergency personnel during use. For instance, the emergency personnel may be provided with one or more packs, each pack containing a pair of cardiac pads with integrated medication delivery mechanisms. The external portion of the pack may include, for example, an indication of the specific type of medication and dosage that may be delivered when the cardiac pad is applied to the patient. In certain embodiments the emergency personnel may be provided with a plurality of packs of cardiac pads containing a variety of medications and dosages, thus enabling the emergency personnel to select the appropriate medication and dosage for the specific patient in cardiac distress. Such a supply of a plurality of packs of cardiac pads may be particularly useful, for example, with a minimalist AED system as provided in U.S. Pat. No. 11,529,526 referenced above.

AED 300 is considered a portable AED due to its size, weight, self-contained power source, and self-contained operations block. In some embodiments, portable AED 300 may have dimensions less than 7 inches in any direction. In some embodiments, portable AED 300 may fit inside of a box having approximately 6.3 by 6.1 by 1.7 inch dimensions. The weight of AED 300 may be two pounds or less, and may be between approximately 1 and 1.5 lbs.

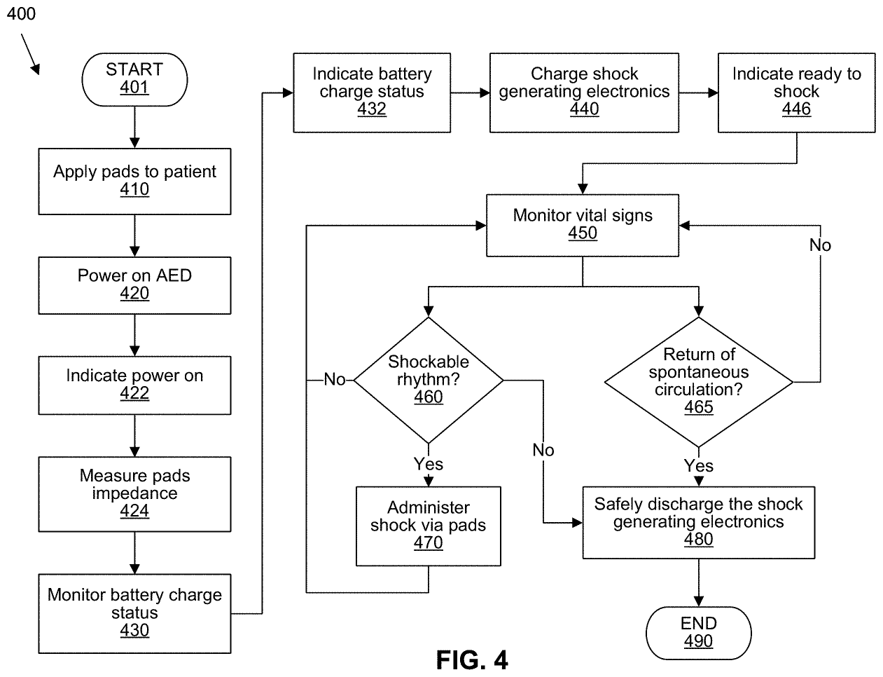

FIG. 4 provides an exemplary AED operation method 300 which is configured for use with AED 10 of FIG. 1, AED 100 of FIG. 2, AED 300 of FIG. 3, or the minimalist AED described in U.S. Pat. No. 11,529,526 referenced above, for example. Method 400 is configured for use by a trained operator such as a certified emergency responder.

Method 400 starts at a step 401. In a step 410, cardiac pads are applied to the patient in cardiac distress. In an example, the user may remove the cardiac pads from a pouch or a cartridge, activate the medicine delivery mechanism (e.g., peel off a protective cover from a transdermal patch or microneedle array integrated into the cardiac pad), then apply the cardiac pads to specified locations on the body of the patient, thus initiating the medicine delivery. In another example of step 410, the user may retrieve the cardiac pads (e.g., from a pocket, storage container, or other location), plug the cardiac pads into the AED, then apply the cardiac pads to specified locations on the body of the patient.

In an optional step 420, the AED is manually powered on. In certain AEDs, the removal of the pads cartridge from the AED enclosure (e.g., such as pads cartridge 310 shown in FIG. 3) automatically turns on the AED, such that step 420 is not needed. In an example of step 420, the AED may be manually powered on via a power-on button or switch. In some embodiments, plugging in the cardiac pads into the AED automatically powers on the AED.

In an optional step 422, an indication is provided that the AED is powered on. The indication may include an audible sound (e.g., a beep) and/or a visual indication (e.g., an illuminated light), thus confirming to a user that the AED has been powered on. Optionally, the AED may automatically perform additional self-checks when the AED is powered on. The self-checks may include a state of the cardiac pads (dry, old, used, etc.), a battery level, an electrical circuitry status, or a software/firmware version, for example.

In an optional step 424, an impedance between the cardiac pads is measured to assess the proper engagement of the pads on the patient's body. In an example of step 424, an impedance between the cardiac pads is acquired by the AED controller (e.g., controller 110 of FIG. 2). When the cardiac pads are placed on a patient's body, the medicine delivery mechanism integrated into the pads is activated, and the impedance measurement relates to the patient's size, and may be used for determining an amount of electrical shock to deliver to the patient. Data regarding body impedance is used to calculate and adjust the appropriate shock waveform via, for example, controller 110. For example, as shown in FIG. 2, the energy output from shock generating electronics 124 may be adjusted according to the body impedance to produce a waveform according to the accepted standard biphasic pattern used in modern defibrillators. In certain embodiments, the voltage waveform is generally between 120 and 200 Joules in total energy. In some embodiments, optional step 424 is omitted from method 400 and the voltage waveform that is produced is based on an average sized adult.

In a step 430, a battery charge status in monitored. In an example of step 230, power management block 130 monitors a charge status of battery 132 of AED 100, as described above in connection with FIG. 2. In embodiments, power management block 130 does not provide continuous monitoring of battery 132. Instead, only periodic monitoring is provided when AED 100 is turned on, and no monitoring is provided when AED 100 is turned off to conserve charge of battery 132.

In an optional step 432, an indication of battery charge status is provided. In an example of step 432, controller 110 of FIG. 2 determines a charge status of battery 132 via power management block 130 and sends a signal indicative of the charge status to UI 150, which indicates the charge status via a charge status indicator.

In a step 440, shock generating electronics are charged. In an example of step 440, controller 110 sends a signal to shock generating electronics 124 to initiate charging from battery 132. In embodiments, shock generating electronics 124 include one or more capacitors configured to store an electrical charge for at least one electrical defibrillation. Shock generating electronics 124 may further include a biphasic truncated exponential power stage, as described in the aforementioned U.S. Pat. No. 11,103,718, which is incorporated by reference in its entirety.

In an optional step 446, a ready-to-shock indication is provided. In an example of step 446, controller 110 determines a status of shock generating electronics 124 and sends a signal indicative of the charge status to UI 150. When the charge status is sufficient, UI 150 then provides an audio or visual indication that the AED 100 is ready to administer an electrical shock to the patient. The audio indication may be a beep or series of beeps or a voice command, for example. The visual indication may be a light or an illuminated or blinking light, or an array of lights, for example.

In a step 450, vital signs are monitored. In an example of step 450, a patient's vital signs are monitored via cardiac pads. In embodiments, ECG signals are transmitted from cardiac pads 122 to ECG monitoring circuitry 120, which is communicatively coupled with controller 110. Controller 110 monitors the patient's ECG pattern and determines a status of the patient's vital signs. Step 450 may be performed while the shock generating electronics are simultaneously charging in step 440.

In a step 460, a shockable rhythm is determined. In an example of step 460, controller 110 receives ECG signals from cardiac pads 122 and differentiates between “shockable” rhythms and “unshockable” patterns. An associated algorithm may run internally within controller 110 without real-time access to the cloud, or to any attached device such as a smartphone. Such an algorithm is defined, in the present disclosure, as a shock indicator algorithm (SIA). The specific conditions required for differentiation between shockable and unshockable cardiac rhythms, which are identified by the SIA, follow guidance from industry organizations. In an embodiment, the SIA is prioritized above other processing activities within controller 110 such that the SIA interrupts any other processes in controller 110 to commence the shock protocol, to the exclusion of other activities.

When analysis by controller 110 determines that the cardiac rhythm detected is a shockable rhythm, and when the shock generating electronics 124 have been charged, method 400 then proceeds to a step 470 to administer a shock to the patient.

In step 470, a shock is administered via the cardiac pads. In an example of step 470, an electrical shock is administered to a patient via cardiac pads 122. The electrical shock may be a biphasic waveform with a precise shape according to precise timing specifications. In some embodiments, delivery of the shock to the patient is automated such that upon charge completion of the shock generating electronics in step 440 combined with a shockable rhythm being detected in step 460, controller 110 instructs AED 100 to deliver the shock. In some embodiments, delivery of the shock to the patient may be manually initiated by the user by pressing a button (e.g., second button 332) or speaking a voice command that is received by controller 110 via the optional microphone. Alternatively, AED 100 is configured for telemedicine and delivery of the shock to the patient is initiated by a remote user via a tethered device (e.g., a smartphone).

Following step 470, method 400 returns to step 430 to monitor the battery charge status. If the battery charge status is sufficient, method 400 may proceed to optional step 432 to provide a notification of battery charge status, to step 440 to charge the shock generating electronics, and to step 450 to monitor vital signs, in preparation for performing another shock if necessary.

Alternatively in step 460, if analysis by controller 110 determines that the cardiac rhythm detected is an unshockable cardiac rhythm, method 400 returns to step 450 to monitor vital signs for a predetermined duration or until the method is ended by a user. For example, AED 100 may remain powered on with the shock generating electronics 124 charged while awaiting further instructions for the predetermined duration (e.g., five or ten minutes). If the method is ended, either at the end of the predetermined duration or by the user, method 400 proceeds to a step 480 to safely discharge the shock generating electronics and to a step 490 to end.

In a step 465, the controller 110 determines if return of spontaneous circulation (ROSC) has occurred. While monitoring vital signs in step 450, if ROSC is detected via the SIA, controller 110 determines that a shock is not needed and method 400 may proceed to step 480 to safely discharge the shock generating electronics and to step 490 to end.

In step 480, the shock generating electronics are safely discharged. In an example of step 480, the shock generating electronics 124 are safely discharged via an internal discharge circuit (e.g., as part of power management block 130) by activating a power resister.

In step 490, method 400 ends. In an example of step 490, AED 100 may automatically power off to conserve battery power. Alternatively, AED 100 may proceed to a standby mode in which the power remains on for a predetermined duration before powering off. For example, AED 100 may remain powered on for one, five, or ten minutes before powering off. A user may turn off AED 100 at any time by pressing and holding the power button for a predetermined duration (e.g., three seconds). In an embodiment, disconnecting (e.g., unplugging) cardiac pads 122 may automatically turn off AED 100 after a predetermined duration in a standby mode.

Steps of method 400 may be performed in the order shown in FIG. 4, or the order of steps may be modified without departing from the scope hereof. It is emphasized that method 400 provides both medicine delivery and AED shock delivery at the same time, without adding steps beyond those required for the AED shock delivery. In other words, method 400 provides both medicine delivery and electric shock delivery without the introduction of additional steps for the medicine delivery, thus saving time and effort for the emergency responder.

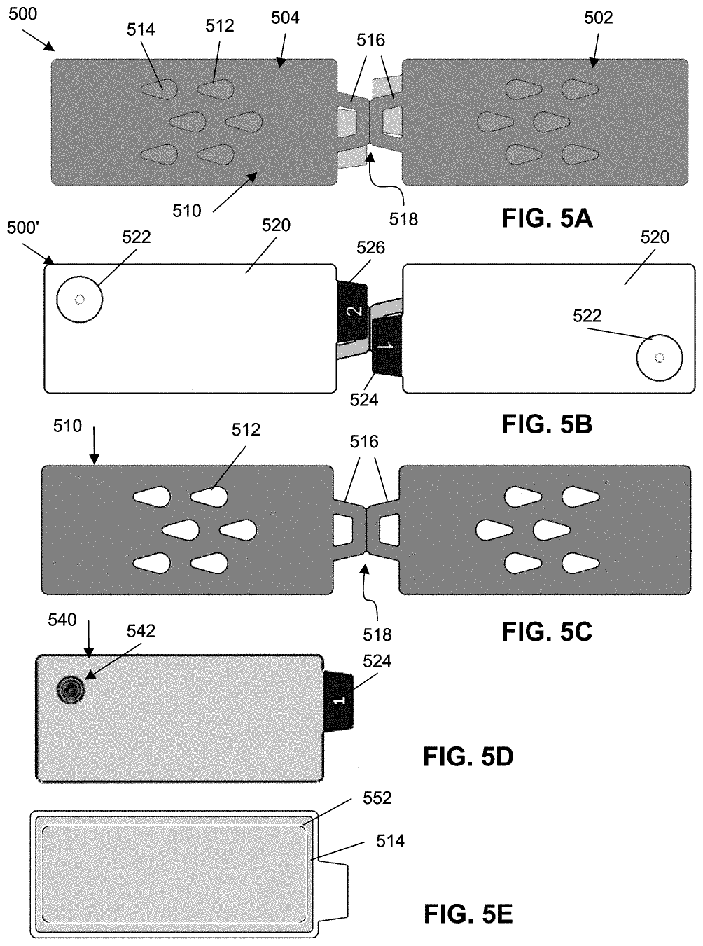

FIGS. 5A-5E illustrate an exemplary embodiment of pads suitable for use with the AED system with medicine delivery, as described above. FIG. 5A shows a liner-side view of a pads assembly 500 including a first cardiac pad 502 and a second cardiac pad 504, in accordance with an embodiment. As shown in FIG. 5A, pads assembly includes a liner 510 with a plurality of holes 512 formed therein, through which portions of an adhesive layer 514 is visible. Liner 510 includes a pair of tabs 516 connected at a connection 518 such that liner 510 serves to keep first and second cardiac pads 502 and 504 aligned and attached to each other when stored, such as in an electrostatic discharge (ESD)-protected pouch (not shown). In embodiments, connection 518 includes a perforated section such that tabs 516 may be separated by a user. In embodiments, liner 510 is configured to be foldable such that, when not in use, pads assembly 500 may be folded at connection 518. In certain embodiments, holes 512 on the first and second cardiac pads are configured to be aligned when folded to reduce the likelihood of adhesive layer 514 becoming dried out during storage.

FIG. 5B shows an electrode-side view 500′ of the pads assembly, in accordance with an embodiment. As visible in FIG. 5B, the pads assembly further includes an electrode protector 520 on each one of first and second cardiac pads 502 and 504 to enclose the electrode assemblies contained therein, thus protecting the user and the patient from errant electrical shock. The pads assembly may also include connector covers 522 to protect the wiring (not shown) used in electrically connecting the pads assembly with the shock-generating electronics of the AED. Further, first and second tabs 524 and 526 protrude respectively from first and second cardiac pads, respectively, to assist with the correct placement of the first and second cardiac pads on a patient.

FIG. 5C shows liner 510 in isolation. FIG. 5D shows an electrode assembly 540 corresponding to first cardiac pad 502, including an electrode set 542, shown here as a dark circular feature including circular washer and eyelet combination for attaching wires thereto (not shown), which provides the shock delivery from the AED. The electrode set optionally may further include circuitry (e.g., RFID chip, NIR chip, or the like) to indicate to the AED the type and dosage of the integrated medication, as described above, such that the AED is able to identify the specific details of the medication contained in the particular cardiac pads.

FIG. 5E shows the liner-side view of first electrode 502 with the liner removed. As visible in FIG. 5E, adhesive layer 514 is configured to encase a conductive layer (represented by a dashed line 552) to conduct the electrical shock from the AED via the electrode set (not visible in FIG. 5E). The adhesive layer further includes, in an example, a hydrogel layer impregnated with a medication, such as naloxone. The adhesive layer then operates as a transdermal patch for providing the impregnated medication to the patient as soon as the pads are attached to the patient. Alternatively, the adhesive layer may also be integrated with microneedle structures so as to deliver the medication via microneedles.

It is noted that assembly of cardiac pads assembly 500 is substantially the same as the assembly process for currently available commercial cardiac pads. A primary distinction is the integration of a medication delivery mechanism, such as a transdermal patch or microneedle structures for the delivery of medication to the patient upon attachment of the cardiac pads. Further, optional features, such as the medication-identification circuitry described above, additional labeling on the liner 510, electrode protector 510, or even adhesive layer 514 itself to identify the specifics of the medication contained therein, such as the name, composition, and dosage.

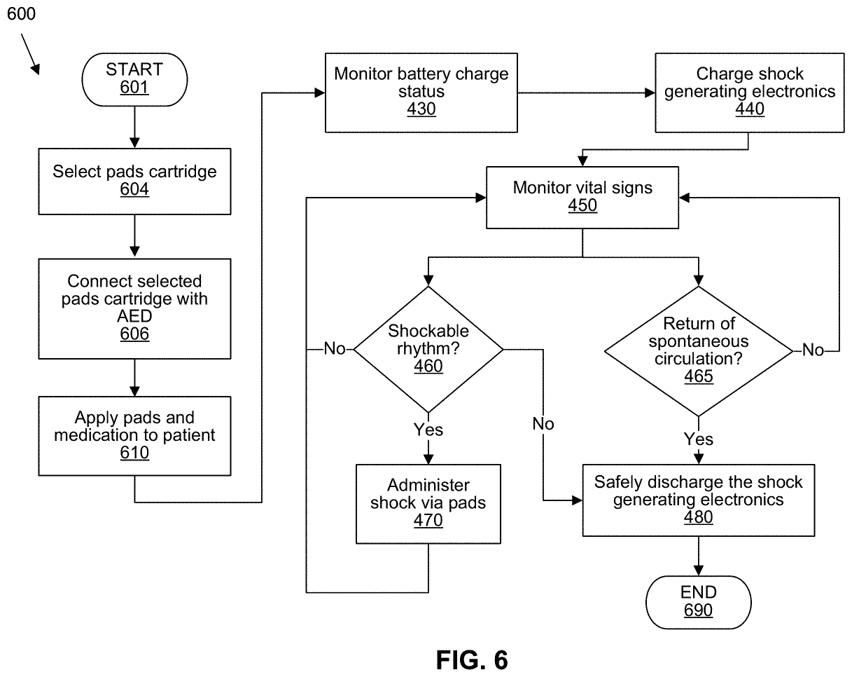

FIG. 6 shows an alternative method for operating an AED suitable for use with cardiac pads with medication delivery. A method 600 includes several of the same steps as method 400 of FIG. 4, particularly related to steps 430 through 480 related to the shock delivery. The various optional steps included in method 400 of FIG. 4 are also omitted in FIG. 6 for illustrative clarity. Method 600 begins with a start step 601 and further includes a step 604 to select the desired pads cartridge, such as the specific pads cartridge pack including the appropriate medication and dosage for the specific patient being treated. Method 600 also includes a step 606 to connect the selected pads cartridge with the AED, then applying the pads, with medication delivery, to the patient in a step 610. Then method 600 proceeds to similar shock delivery steps as shown in FIG. 4, then terminates in an end step 690.

It is emphasized that both method 400 of FIG. 4 and method 600 of FIG. 6 provide essentially identical protocols to the normal operations of an AED used to treat a patient in cardiac distress. With the integration of the medication delivery mechanism into the cardiac pads used in the shock delivery from the AED, methods 400 and 600 enable automatic delivery of additional medication to the patient without significantly modifying or interrupting the life-saving protocol for electrical shock delivery.

Features described above as well as those claimed below may be combined in various ways without departing from the scope hereof. The following examples illustrate some possible, non-limiting combinations:

(A1) An automated external defibrillator (AED) system includes shock generating electronics configured to provide at least one electrical shock suitable for a patient experiencing a cardiac event, a battery configured for providing power to the shock generating electronics, power management circuitry configured for managing the shock generating electronics and the battery, a single microprocessor configured for controlling the power management circuitry, an enclosure configured to house the shock generating electronics, the battery, the power management circuitry, and the single microprocessor, and a pair of cardiac pads configured for electrical connection with the shock generating electronics. At least one of the cardiac pads in the pair of cardiac pads includes a medication delivery mechanism configured for delivering a predetermined dose of a medication to a patient when the cardiac pads are placed on the patient for shock delivery.

(A2) For the AED system denoted as (A1), the medication delivery mechanism may include at least one of a medication-impregnated transdermal patch, and a microneedle array.

(A3) For the AED system denoted as (A1) or (A2), the medication may include naloxone.

(A4) For the AED system denoted as any of (Al) through (A3), a plurality of pairs of cardiac pads may be provided with each one of the plurality of pairs of cardiac pads being configured for electrical connection with the shock generating electronics one at a time, and each one of the plurality of pairs of cardiac pads may include a different predetermined dose of medication from each other one of the plurality of pairs of cardiac pads.

(A5) For the AED system denoted as any of (A1) through (A4), a clip may be mounted to an exterior of the enclosure and the clip may be configured for clipping the AED system to a location for carrying the AED system.

(A6) For the AED system denoted as any of (A1) through (A5), at least one of the cardiac pads in the pair of cardiac pads may include a medication identification feature for identifying the medication integrated thereon.

(A7) For the AED system denoted as any of (A1) through (A6), the medication identification feature may include at least one of a radio frequency identification (RFID) chip, a near infrared (NIR) chip, an identification circuit, a liner label, an electrode protector label, an adhesive label, and a package label.

(B1) A method for using an automated external defibrillator (AED) system to apply an electrical shock to a patient experiencing a cardiac event, the AED system including shock generating electronics and a pair of cardiac pads connected with the shock generating electronics, the method includes: applying the cardiac pads to the patient, and delivering the electrical shock simultaneously with a medication to the patient via the cardiac pads. Each one of the cardiac pads includes a delivery mechanism for administering a medication to the patient such that applying the cardiac pads to the patient includes administering the medication to the patient simultaneously with delivering the electrical shock to the patient.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

What is claimed is:

1. An automated external defibrillator (AED) system comprising:
shock generating electronics configured to provide at least one electrical shock suitable for a patient experiencing a cardiac event;
a battery configured for providing power to the shock generating electronics;
power management circuitry configured for managing the shock generating electronics and the battery;
a single microprocessor configured for controlling the power management circuitry;
an enclosure configured to house the shock generating electronics, the battery, the power management circuitry, and the single microprocessor; and
a pair of cardiac pads configured for electrical connection with the shock generating electronics,
wherein at least one of the cardiac pads in the pair of cardiac pads includes a medication delivery mechanism configured for delivering a predetermined dose of a medication to a patient when the cardiac pads are placed on the patient for shock delivery.

2. The AED system of claim 1, wherein the medication delivery mechanism includes at least one of a medication-impregnated transdermal patch, and a microneedle array.

3. The AED system of claim 2, wherein the medication includes naloxone.

4. The AED system of claim 1, further comprising a plurality of pairs of cardiac pads, each one of the plurality of pairs of cardiac pads being configured for electrical connection with the shock generating electronics one at a time, and
wherein each one of the plurality of pairs of cardiac pads includes a different predetermined dose of medication from each other one of the plurality of pairs of cardiac pads.

5. The AED system of claim 1, further comprising a clip mounted to an exterior of the enclosure, wherein the clip is configured for clipping the AED system to a location for carrying the AED system.

6. The AED system of claim 1, wherein at least one of the cardiac pads in the pair of cardiac pads includes a medication identification feature for identifying the medication integrated thereon.

7. The AED system of claim 6, wherein the medication identification feature includes at least one of a radio frequency identification (RFID) chip, a near infrared (NIR) chip, an identification circuit, a liner label, an electrode protector label, an adhesive label, and a package label.

8. A method for using an automated external defibrillator (AED) system to apply an electrical shock to a patient experiencing a cardiac event, the AED system including shock generating electronics and a pair of cardiac pads connected with the shock generating electronics, the method comprising:
applying the cardiac pads to the patient; and
delivering the electrical shock simultaneously with a medication to the patient via the cardiac pads,
wherein each one of the cardiac pads includes a delivery mechanism for administering a medication to the patient such that applying the cardiac pads to the patient includes administering the medication to the patient simultaneously with delivering the electrical shock to the patient.
